(19) United States
(12) Patent Application Publication
(10) Pub. No.: US 2022/0071899 A1
(43) Pub. Date: Mar. 10, 2022

(54) DRINKABLE INFANT COMPOSITIONS

(71) Applicant: SOCIETE DES PRODUlTS NESTLE S.A., Vevey (CH)

(72) Inventors: Natalie Jaecklein, Grosshochstetten (CH); Martinas Kuslys, Grosshoechstetten (CH); Sophie Nutten, Palezieux-Village (CH); Anne Thevenier, Bern (CH)

(21) Appl. No.: 17/416,580

(22) PCT Filed: Dec. 20, 2019
(86) PCT No.: PCT/EP2019/086830

§ 371 Date: Jun. 21, 2021

(30) Foreign Application Priority Data
Dec. 21, 2018 (EP) 18215009.4

(51) Int. Cl. A61K 9/00; A61K 38/17; A23L 33/00; A23L 33/19; A23L 3/16

(52) U.S. Cl. CPC A61K 9/0095; A61K 38/1709; A23V 2002/00; A23L 33/19; A23L 3/16; A23L 33/40

(57) ABSTRACT

A drinkable infant composition comprising two or more food allergens wherein one of said food allergens is milk protein.

### FIELD OF THE INVENTION

The present invention relates to drinkable infant compositions comprising food allergens. The present invention also relates to methods for producing drinkable infant compositions and the use of drinkable infant compositions for reducing sensitisation to food allergens.

### BACKGROUND TO THE INVENTION

In Europe and North America, food allergy is estimated to affect nearly 5% of adults and 8% of children (Sicherer, S. H. and Sampson, H. A., 2014. Journal of Allergy and Clinical Immunology, 133(2), pp. 291-307). In Australia, a population-based study found a prevalence of challenge-proven food allergy of over 10% which is the highest rate globally (Osborne, N.J., et al., 2011. Journal of Allergy and Clinical Immunology, 127(3), pp. 668-676). Overall, prevalence figures for food allergy and anaphylaxis appear to be steadily rising, with the greatest increase observed in infants with food allergies or atopic eczema (Koplin, J. J., et al., 2015. Current opinion in allergy and clinical immunology, 15(5), pp. 409-416). Effective allergy prevention, particularly for infants, has therefore become a global public health priority (Ring, J., 2012. Allergy, 67(2), pp. 141-143).

Nutritional interventions play a central role in the prevention and treatment of food allergies. In recent years, clinical approaches have undergone significant changes and greater focus has been placed on the early introduction of the complementary diet in infancy (Du Toit, G., et al., 2016. Allergology International, 65(4), pp. 370-377). For instance, The European Society for Paediatric Gastroenterology, Hepatology, and Nutrition (ESPGHAN) Committee on Nutrition now recommends allergenic foods may be introduced when complementary feeding is commenced any time after 4 months (Fewtrell, M., et al., 2017. Journal of pediatric gastroenterology and nutrition, 64(1), pp. 119-132). For example, it is recommended that infants at high risk of peanut allergy (those with severe eczema, egg allergy, or both) should have peanut introduced between 4 and 11 months. Thus, many scientists accept the principle of early introductions of food allergens.

The challenges to introduce food allergens at an early age, for instance before 4 months, are format and safety related.

For instance, common food allergens such as egg, peanut, tree nuts, fish, crustaceans, shellfish and sesame are typically in the format of solid foods which are not suitable for babies from a safety point of view. However, babies at an early stage are not able to eat solid food. During the Enquiring About Tolerance (EAT) study, 6 common food allergens were introduced in the form of solid food to infants from 3 months of age alongside breastfeeding (Perkin, M. R., et al., 2016. Journal of Allergy and Clinical Immunology, 137(5), pp. 1477-1486). However, although the study demonstrated a protective benefit per protocol, the study overall failed on intention-to-treat analysis due to a large proportion of participants who were unable to adhere to the study regimen, in part due to the format of the allergens. Thus, it would be advantageous to consume the food allergens in a liquid format.

The liquid format should fulfil safety requirements as valid for infant formulas. The main concerns are microbiological qualities and contaminants. Thermal denaturation during the pasteurisation or sterilisation of the drinkable infant composition may affect the immunogenic properties of the allergens.

Thus, there is a demand for commercially-available drinkable infant compositions comprising food allergens, which may be administered to young infants.

### SUMMARY OF THE INVENTION

According to the present invention, there is provided a drinkable infant composition comprising two or more food allergens wherein one of said food allergens is milk protein.

The drinkable infant composition may be ingested in a liquid format. Thus, in some preferred embodiments the composition is in a powdered form, preferably wherein the powdered form can be reconstituted with water. In other preferred embodiments the composition is in a ready to drink form.

Gentle pasteurisation and/or sterilisation may be used to provide the drinkable infant composition comprising food allergens. Reducing holding temperatures and/or holding times during heat treatment can be used to reduce or minimise thermal degradation of the allergens.

In one embodiment the drinkable infant composition is pasteurised. In some embodiments the drinkable infant composition has undergone pasteurisation at a temperature of between 61.9° C. and 65° C., preferably between 62° C. and 64° C., preferably wherein the pasteurisation is performed for at least 30 minutes or at least 35 minutes.

In some embodiments the drinkable infant composition is sterilised. Sterilisation may be by indirect or direct ultra-high temperature heat treatment. In some embodiments the drinkable infant composition has undergone an indirect ultra-high temperature heat treatment, preferably at a temperature of between 125° C. and 135° C., or between 130° C. and 134° C., or between 131° C. and 133° C., for example at about 132° C. The sterilisation may be performed, for at least 30 seconds, or at least 60 seconds, for example between 30 and 80 seconds, or 60 to 70 seconds.

In some embodiments the drinkable infant composition has undergone direct ultra-high temperature heat treatment, preferably at a temperature of between 136° C. and 140° C. for about 15 to 25 seconds, for example about 20 seconds, or at a temperature of between 140° C. and 144° C. for about 5 to 15 seconds, for example 10 seconds. In another embodiment the direct UHT heat treatment may be at a temperature of between 150° C. and 154° C., for about 2 to 4 seconds.

In some embodiments the sterilisation may be an ultra short sterilization (USS). In some embodiments, the drinkable infant composition has undergone ultra short sterilization (USS) heat treatment at a temperature of between 155° C. and 170° C., for less than 1 second.

In some embodiments the gentle heat-treatment may be carried out a temperature of between 72° C. and 90° C., for example between 72° C. and 80° C. for 10 to 30 seconds, or between 80° C. and 89° C. for 2 to 20 seconds, for example between 80° C. and 84° C. for 4 20 seconds, or between 85° C. and 89° C. for 1 to 10 seconds.

In some embodiments the drinkable infant formula composition, or the milk protein containing component of the infant formula composition, may undergo a microfiltration step prior to gentle heat treatment.

The composition may contain, for example, 0.01 to 0.03 g/ml of said food allergens. In some embodiments the total amount of said food allergens in the composition is between 0.5 and 5 grams per serving, preferably wherein a serving volume is 15 to 250 ml.

The food allergens may be any known food allergen. Preferably the food allergen is selected from the group consisting of: milk, eggs, cereals (wheat, rye, barley, oats) protein, soybeans, peanuts, tree nuts (including almonds, hazelnuts, walnuts, cashews, pecan nuts, Brazil nuts, pine nuts, pistachio nuts, macadamia nuts), fish, crustaceans, shellfish, celery and celeriac, mustard and sesame. In some embodiments the composition comprises three or more of said food allergens, or four or more of said food allergens, or five or more of said food allergens, or six or more of said food allergens, or seven or more of said food allergens, or eight or more of said food allergens, or nine or more of said food allergens or all of said food allergens. In one embodiment the drinkable infant composition comprises milk protein and egg protein.

The composition may be for use in infants between 0-12 months, 6 weeks to 12 months, 0-6 months, 6 weeks to 6 months, 0-4 months or 6 weeks to 4 months of age. In an embodiment the composition is for use in infants between about 1 month to about 8 months, such as about 1 month to about 7 months, or about 1 month to about 6 months. In an embodiment the composition is for use in infants between about 1 month and about 4 months, or between about 1 month and about 3 months.

The drinkable infant composition may further comprise one or more carriers, preferably wherein the carrier is skimmed milk powder and/or lactose.

The drinkable infant composition may further comprise a probiotic, and/or a human milk oligosaccharide.

In one embodiment at least 20%, preferably at least 30% of the food allergens are non-denatured.

In one aspect the present invention provides a drinkable infant composition as defined herein for use in reducing or preventing allergies to said food allergens in infants.

In another aspect the present invention provides a method of reducing or preventing allergies in infants by administering an effective amount of a drinkable infant composition as defined herein.

In another aspect the present invention provides a process for producing a drinkable infant composition as defined herein, comprising the steps:
i) blending two or more food allergens wherein one of said allergens is milk protein to form a mixture;
ii) homogenising said mixture;
iii) pasteurising said mixture;
iv) optionally spray-drying said mixture;
   preferably wherein pasteurisation is performed at a temperature of between 61.9° C. and 65° C., preferably between 62° C. and 64° C., preferably wherein the pasteurisation is performed for at least 30 minutes or at least 35 minutes.

In another aspect the present invention provides a process for producing a drinkable infant composition as defined herein, comprising the steps:
i) blending two or more food allergens wherein one of said allergens is milk protein to form a mixture;
ii) homogenising said mixture;
iii) sterilising said mixture;
iv) optionally spray-drying said mixture;

The sterilisation may be an indirect ultra-high temperature heat treatment as described herein. The sterilisation may be a direct ultra-high temperature heat treatment as described herein.

### DETAILED DESCRIPTION

The terms “comprising”, “comprises” and “comprised of” as used herein are synonymous with “including” or “includes”; or “containing” or “contains and are inclusive or open-ended and do not exclude additional, non-recited members, elements or steps. The terms “comprising”, “comprises” and “comprised of” also include the term “consisting of”.

### Drinkable Infant Composition

As used herein, the term “drinkable infant composition” means a composition suitable for consumption by an infant. Examples of drinkable infant compositions include, but are not limited to, infant supplements and infant formulas.

During the first four months of age infants are usually unable to consume solid foods. Thus, introduction of food allergens via solid foods is not possible and it would be advantageous to consume the food allergens in a liquid format. Accordingly, in preferred embodiments the drinkable infant composition may be provided and/or ingested in a liquid format. In some embodiments the drinkable infant composition is for use in infants who are not able to eat solid food.

In a preferred embodiment the drinkable infant composition is an infant supplement. The supplement may be provided in addition to breast milk and/or infant formula. Thus, in a preferred embodiment the drinkable infant composition is an infant supplement suitable for ingestion by infants from 0-12 months, 6 weeks to 12 months, 0-6 months, 6 weeks to 6 months, 4-6 months, preferably 0-4 months or 6 weeks to 4 months of age. In an embodiment the composition is for use in infants between about 1 month to about 8 months, such as about 1 month to about 7 months, or about 1 month to about 6 months. In an embodiment the composition is for use in infants between about 1 month and about 4 months, or between about 1 month and about 3 months.

The infant supplement may further comprise one or more of (in addition to that provided by the source of food allergens): protein; fat (lipids); carbohydrates; and essential vitamins and minerals. The infant supplement may further comprise sweetening, flavouring and/or colouring agents.

In other embodiments the drinkable infant composition does not comprise additional protein; fat; carbohydrates; and/or essential vitamins and minerals. For example, in some embodiments the drinkable infant composition does not comprise any components other than those provided by the source of the food allergens and optionally the one or more carriers, if included.

In other embodiments the drinkable infant composition is an infant formula or follow-on formula. The expression “infant formula” means a foodstuff intended for particular nutritional use by infants during the first four to six months of life and satisfying by itself the nutritional requirements of this category of person. The expression “follow-on formula” means a foodstuff intended for particular nutritional use by infants aged over four months and constituting the principal liquid element in the progressively diversified diet of this category of person.

Requirements for infant formula are well known to those of skill in the art. For instance, recommendations and requirements are provided by The European Society for Pediatric Gastroenterology, Hepatology and Nutrition (ESPGHAN) e.g. Koletzko, B., et al., 2005. “Global standard for the composition of infant formula: recommendations of an ESPGHAN coordinated international expert group” Journal of pediatric gastroenterology and nutrition, 41(5), pp. 584-599. Typically, an infant formula in a ready-to-consume liquid form (for example reconstituted from a powder) provides 60-70 kcal/100 ml. Infant formula typically comprises, per 100 kcal: about 1.8-4.5 g protein; about 3.3-6.0 g fat (lipids); about 300-1200 mg linoleic acid; about 9-14 g carbohydrates selected from the group consisting of lactose, sucrose, glucose, glucose syrup, starch, maltodextrins and maltose, and combinations thereof; and essential vitamins and minerals.

In some embodiments the drinkable infant composition further comprises one or more carriers. As used herein the term “carrier” is any substance useful as an excipient, filler, bulking agent, diluent, colouring agent, stabiliser, thickener, binder, flavouring agent and the like. Preferably the one or more carriers comprise skimmed milk powder and/or lactose. Most preferably the carrier is skimmed milk powder and/or lactose.

In some embodiments the drinkable infant composition further comprises a probiotic, and/or a human milk oligosaccharide (e.g. prebiotic) and/or a postbiotic.

The term “probiotic” refers to microbial cell preparations or components of microbial cells with beneficial effects on the health or well-being of the host (Salminen, S. et al. (1999) Trends Food Sci. Technol. 10: 107-10).

In particular, probiotics may improve gut barrier function (Rao, R. K. (2013) Curr. Nutr. Food Sci. 9: 99-107).

Examples of probiotic micro-organisms for use in the composition of the present invention include yeasts, such as Saccharomyces, Debaromyces, Candida, Pichia and Torulopsis; and bacteria, such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus.

Specific examples of suitable probiotic microorganisms are: Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus and Staphylococcus xylosus.

Exemplary probiotic bacterial strains include Lactobacillus rhamnosus; Lactobacillus rhamnosus LPR (CGMCC 1.3724); Bifidobacterium lactis BL818 (CNCM 1-3446) sold inter alia by the Christian Hansen company of Denmark under the trade mark BB 12; and Bifidobacterium longum BL999 (ATCC BAA-999) sold by Morinaga Milk Industry Co. Ltd. of Japan under the trade mark BB536.

Prebiotics are usually non-digestible in the sense that they are not broken down and absorbed in the stomach or small intestine and thus remain intact when they pass into the colon where they are selectively fermented by the beneficial bacteria. Examples of prebiotics include certain oligosaccharides, such as fructooligosaccharides (FOS), inulin, xylooligosaccharides (XOS), polydextrose or any mixture thereof. In a particular embodiment, the prebiotics may be fructooligosaccharides and/or inulin. An example is a combination of 70% short chain fructooligosaccharides and 30% inulin, which is registered by Nestle under the trademark “Prebio 1”.

In a particular embodiment the prebiotic(s) may be human milk oligosaccharide(s). Human milk oligosaccharides (HMOs) are, collectively, the third largest solid constituents in human milk, after lactose and fat. HMOs usually consists of lactose at the reducing end with a carbohydrate core that often contains a fucose or a sialic acid at the non-reducing end. There are approximately one hundred milk oligosaccharides that have been isolated and characterized. Many infant formulae have been developed using HMO ingredients, such as fucosylated oligosaccharides, lacto-N-tetraose, lacto-N-neotetraose, or sialylated oligosaccharides, for different purposes.

Postbiotics are non-viable bacterial products or metabolic by-products from probiotic microorganisms that have biologic activity in the host. Exemplary postbiotics include bioactive components produced during fermentation such as short chain fatty acids, enzymes, peptides, polysaccharides, cell surface proteins or vitamins. Postbiotics can support immune function through the gut.

The drinkable infant composition may be in a powdered form, wherein the powdered form can be reconstituted into a liquid format prior to ingestion. In preferred embodiments the drinkable infant composition is reconstituted with water prior to ingestion. Preferably the composition may be reconstituted with water to provide one serving.

In other preferred embodiments the composition is in a ready to drink form. The ready to drink composition may be provided in a bottle. Preferably the bottle provides one serving of food allergens.

As used herein the term “serving” means a recommended portion to be ingested by one infant in one feed, also known as a single dose. A person skilled in the art will be aware that the size of a specific serving or dose will depend on a variety of factors including age, body weight, general health, diet and time of administration. Thus, in some embodiments the serving may provide 0.5 to 5 g, 1 to 4 g or 2 to 3 g of total food allergens. The serving may comprise, for example, 15 to 250 ml, 35 to 200 ml, 50 to 150 ml, 75 to 125, 10 to 100 ml, 50 to 90 ml, 60 to 80 ml or about 70 ml of the drinkable infant composition. In one embodiment, a serving provides 0.5 to 5 g of total food allergens in 15 to 250 ml or 50 to 150 ml.

In some embodiments the drinkable infant composition contains 0.01 to 0.1 g/ml, preferably 0.01 to 0.05 g/ml, or most preferably 0.01 to 0.03 g/ml of total food allergens.

### Food Allergens

As used herein, the term “food allergen” refers to proteins or derivatives thereof that cause abnormal immune responses. Purified food allergens may be named using the systematic nomenclature of the Allergen Nomenclature Sub-Committee of the World Health Organization and International Union of Immunological Societies. Allergen names are composed of an abbreviation of the scientific name of its source (genus: 3-4 letters; species: 1-2 letters) and an Arabic numeral, for example Der p 1 for the first allergen to be described from the house dust mite Dermatophagoides pteronyssinus. Food allergens are derived from proteins with a variety of biologic functions, including proteases, ligand-binding proteins, structural proteins, pathogenesis-related proteins, lipid transfer proteins, profilins, and calcium-binding proteins.

A list of food allergens is provided on the official website of the WHO/IUIS Allergen Nomenclature Database, http://www.allergen.org/index.php. (Radauer, C., et al., 2014. Allergy, 69(4), pp. 413-419 and Pomés, A., et al., 2018. Molecular immunology).

The invention provides a drinkable infant composition comprising two or more food allergens, from different food sources wherein one of said food allergens is milk. Preferably the one or more other food allergens are selected from the group consisting of: eggs, cereals (wheat, rye, barley, oats) protein, soybeans, peanuts, tree nuts (including almonds, hazelnuts, walnuts, cashews, pecan nuts, Brazil nuts, pine nuts, pistachio nuts, macadamia nuts), fish, crustaceans, shellfish, celery and celeriac, mustard and sesame.

In some embodiments the drinkable infant composition comprises three or more of said food allergens (i.e. three or more selected from the group of milk protein, egg protein, wheat protein, soya protein, peanut protein, tree nut protein, fish protein, crustacean protein, shellfish protein, and sesame protein), or four or more of said food allergens, or five or more of said food allergens, or six or more of said food allergens, or seven or more of said food allergens, or eight or more of said food allergens, or nine or more of said food allergens or ten or more of said food allergens.

In one embodiment the drinkable infant composition comprises milk protein and egg protein.

In one embodiment the drinkable infant composition comprises milk protein and peanut protein.

In one embodiment the drinkable infant composition comprises milk protein and tree nut protein.

In one embodiment the drinkable infant composition comprises milk protein and wheat protein.

In one embodiment the drinkable infant composition comprises milk protein and fish protein.

In one embodiment the drinkable infant composition comprises milk protein and soya protein.

In one embodiment the drinkable infant composition comprises milk protein and crustacean protein.

In one embodiment the drinkable infant composition comprises milk protein and shellfish protein.

In one embodiment the drinkable infant composition comprises milk protein and sesame protein.

In one embodiment the drinkable infant composition comprises milk protein, egg protein and peanut protein.

In one embodiment the drinkable infant composition comprises milk protein, egg protein, peanut protein and tree nut protein.

In one embodiment the drinkable infant composition comprises milk protein, egg protein and fish protein.

In one embodiment the drinkable infant composition comprises milk protein, egg protein, peanut protein, tree nut protein and fish protein.

In one embodiment the drinkable infant composition comprises egg protein, wheat protein, soya protein, peanut protein, tree nut protein, fish protein, crustacean protein, shellfish protein and sesame protein.

In one embodiment, the drinkable infant composition does not comprise any further food allergens other than those referred to herein.

In some embodiments the amount of each food allergen will be about the same. For example, a serving of the drinkable infant composition may comprise about 0.01 g to about 1 g of each food allergen, about 0.05 g to about 0.5 g of each food allergen or about 0.1 g to about 0.5 g of each food allergen.

The food allergen e.g. peanut allergen etc. may be a mixture of proteins. In some embodiments the food allergen comprises one or more, two or more, three or more, four or more, substantially all or all allergenic components of said food product. For example, in some embodiments the milk protein comprises one or more, two or more, three or more, four or more, substantially all, or all allergenic proteins derived from milk. Examples of known allergenic proteins for specific food products are well known to those of skill in the art.

### Milk Protein

Cow's milk is the most common source of infant food allergy, affecting 1.4-3.8% of young children (Du Toit, G., et al., 2016. Allergology International, 65(4), pp. 370-377.). It can be IgE-mediated with immediate reactions such as urticaria, angioedema and/or anaphylaxis or non-IgE mediated which often manifests with skin or gastrointestinal symptoms (Du Toit, et al.).

It has been shown that early exposure to cow's milk protein is protective against IgE-mediated cow's milk protein allergy (Katz, Y., et al., 2010. Journal of Allergy and Clinical Immunology, 126(1), pp. 77-82).

Milk allergens (including those that exhibit an IgE-mediated response) are known in the art, for instance they are described in Wal, J. M., 2002. Annals of Allergy, Asthma & Immunology, 89(6), pp. 3-10 and provided by the WHO/IUIS Allergen Nomenclature Database.

The milk protein is preferably cow's milk protein. In some embodiments the milk protein comprises one or more of the proteins selected from the list consisting of alpha-lactalbumin (Bos d 4), beta-lactoglobulin (Bos d 5), bovine serum albumin (Bos d 6), immunoglobulin (Bos d 7), caseins (Bos d 8), including alphaS1-casein (Bos d 9), alphaS2-casein (Bos d 10), beta-casein (Bos d 11) and kappa-casein (Bos d 11).

### Egg Protein

Egg allergy is the second most common food allergy with a prevalence rate of approximately 2.5% (Du Toit, et al.). It has been shown that early egg introduction reduces the prevalence of egg allergy (Perkin, M. R., et al., 2016. Journal of Allergy and Clinical Immunology, 137(5), pp. 1477-1486.) The Enquiring About Tolerance (EAT) study found that egg allergy occurred in 3.7% of the early introduction group compared to 5.4% in the standard introduction group (relative reduction 31%).

Egg allergens are known in the art, for instance they are described in Amo, A., et al., 2010. Journal of agricultural and food chemistry, 58(12), pp. 7453-7457 and provided by the WHO/IUIS Allergen Nomenclature Database.

The egg protein may be hen egg protein. In some embodiments the egg protein comprises one or more of the proteins selected from the list consisting of ovomucoid (Gal d 1), ovoalbumin (Gal d 2), ovotransferrin (Gal d 3), lysozyme C (Gal d 4). In some embodiments the egg protein comprises one or more of the proteins selected from the list consisting of ovomucoid (Gal d 1), ovalbumin (Gal d 2), ovotransferrin (Gal d 3), lysozyme C (Gal d 4), alpha-livetin/serum albumin (Gal d 5), yolk glycoprotein 42 (YGP42, Gal d 6), Myosin light chain 1f (Gal d 7), alpha-parvalbumin (Gal d 8), Beta-enolase (Gal d 9), Aldolase (Gal d 10).

### Peanut Protein

Although the prevalence of peanut allergy is less common than milk or egg allergy, it can induce life-threatening anaphylaxis. The results from the Learning Early about Peanut Allergy (LEAP) study demonstrated that in this cohort of high-risk atopic children, early introduction and regular ongoing consumption of peanut resulted in a significant reduction (81% relative reduction, intention to treat analysis) in the number of children with peanut allergy at 60 months of age compared to those who avoided peanut (Du Toit, G., et al., 2013. Journal of Allergy and Clinical Immunology, 131(1), pp. 135-143). The EAT study also demonstrated that the prevalence of peanut allergy (0% vs 2.5%, p=0.003) was less in the early-introduction group compared to the standard-introduction group.

Peanut allergens are known in the art, for instance they are described in Krause, S., et al., 2009. Journal of Allergy and Clinical Immunology, 124(4), pp. 771-778 and provided by the WHO/IUIS Allergen Nomenclature Database.

In some embodiments the species of peanut is Arachis hypogaea. In some embodiments the peanut protein comprises one or more of the proteins selected from the list consisting of Cupins (Vicillin-type, 7S globulin, Ara h 1 and Legumin-type, 11S globulin, Glycinin, Ara h 3), 2S albumins (Ara h 2, 6, 7),), Profilin (Ara h 5), Pathogenesis-related protein, PR-10 (Ara h 8), Nonspecific lipid-transfer proteins type 1 (Ara h 9, Ara h 16, Ara h 17), oleosins (Ara h 10, Ara h 11, Ara h 14 and 15), Defensins (Ara h 12 and 13).).

### Wheat Protein

It has been reported that delaying initial exposure to cereal grains until after 6 months may increase the risk of developing wheat allergy (Poole, J. A., et al., 2006. Pediatrics, 117(6), pp. 2175-2182.). In the study by Poole et al the prevalence of parent-reported wheat allergy was 1%, with detectable wheat-specific IgE antibodies found in 4 of the children (0.25%). All 4 of these children were first exposed to the cereal grains after 6 months. Thus, early introduction of wheat may decrease the risk of developing wheat allergy.

Wheat allergens are known in the art, for instance they are described in Tatham, A. S. and Shewry, P. R., 2008. Clinical & Experimental Allergy, 38(11), pp. 1712-1726 and provided by the WHO/IUIS Allergen Nomenclature Database.

In some embodiments the species of wheat is Triticum aestivum. In some embodiments the wheat protein comprises one or more of the proteins selected from the list consisting of Non-specific lipid transfer protein 1 (Tri a 14), beta-amylase (Tri a 17), Agglutinin isolectin 1 (Tri a 18), Omega-5 gliadin (Tri a 19), Gamma gliadin (Tri a 20), Thioredoxin (Tri a 25), High molecular weight glutenin (Tri a 26), Low molecular weight glutenin GluB3-23 (Tri a 36), Alpha purothionin (Tri a 37), Mitochondrial ubiquitin ligase activator of NFKB 1 (Tri a 41), Tri a 42 and Tri a 43 (hypothetical proteins from cDNA) Endosperm transfer cell specific PR60 precursor (Tri a 44), Elongation factor 1 (Tri a 45).

### Soya Protein

Soy allergy affects approximately 0.4% of young children in the US (Kattan, J. D. and Sampson, H. A., 2015. The Journal of Allergy and Clinical Immunology: In Practice, 3(6), pp. 970-972). Early introduction of soya may decrease the risk of developing soy allergy.

Soya allergens are known in the art, for instance they are described in Kattan, J. D. and Sampson, H. A., 2015. The Journal of Allergy and Clinical Immunology: In Practice, 3(6), pp. 970-972 and provided by the WHO/IUIS Allergen Nomenclature Database.

In some embodiments the species of soybean is Glycine max. In some embodiments the soya protein comprises one or more of the proteins selected from the list consisting of profilin (Gly m 3), Pathogenesis-related protein, PR-10 (Gly m 4), Beta-conglycinin (Gly m 5), Glycinin (Gly m 6), Seed biotinylated protein (Gly m 7) and 2S albumin (Gly m 8).

### Tree Nut Protein

Early introduction of tree nuts may decrease the risk of developing tree nut allergy (Frazier, A. L., et al., 2014. JAMA pediatrics, 168(2), pp. 156-162.) For instance, Frazier et al. have reported that among mothers without tree nut allergy, higher peripregnancy consumption of tree nut was associated with lower risk of tree nut allergy in their offspring. They suggested that this supports the hypothesis that early allergen exposure increases tolerance and lowers risk of childhood food allergy.

Tree nut allergens are known in the art, for instance they are described in Roux, K. H., et al., 2003. International archives of allergy and immunology, 131(4), pp. 234-244 and provided by the WHO/IUIS Allergen Nomenclature Database.

In some embodiments the species of tree nut is selected from one or more of the group consisting of hazelnut, walnut, cashew, almond, pecan, chestnut, Brazil nut, pine nut, macadamia nut, pistachio, coconut, Nangai nut and acorn. In some embodiments the species of tree nut is hazelnut, walnut, cashew and almond. In some embodiments the tree nut protein comprises one or more of the proteins selected from the list consisting of lipid transfer proteins, profilins, members of the Bet v 1-related family, legumins, vicilins, albumins. In some embodiments the tree nut protein comprises one or more of the proteins selected from the list consisting of Cora 1, 2, 8, 9, 11-14 (hazelnut); Jug n 1, 2, 4 (Black walnut); Jug r 1-8 (English walnut); Ana o 1-3 (cashew); Pru du 3-6 and Pru du 8 (almond); Car i 1, 2, 4 (pecan); Cas s 5, 8, 9 (chestnut); Ber e 1, 2 (Brazil nut); and Pis v 1-5 (pistachio). In some embodiments the tree nut protein comprises one or more of the proteins selected from the list consisting of Cor a 1, 2, 8, 9, 11-14; Jug n 1, 2, 4; Jug r 1-8; Ana o 1-3; Pru du 3-6.

### Fish Protein

Early introduction of fish may decrease the risk of developing fish allergy (Kull, I., et al., 2006. Allergy, 61(8), pp. 1009-1015.) For instance, Kull et al. report that Regular fish consumption before year 1 appears to be associated with a reduced risk of allergic disease and sensitization to food and inhalant allergens during the first 4 years of life.

Fish allergens are known in the art, for instance they are described in Poulsen, L. K., et al., 2001. Allergy, 56, pp. 39-42 and provided by the WHO/IUIS Allergen Nomenclature Database.

In some embodiments the species of fish is selected from one or more of the group consisting of cod, herring, trout, tuna, salmon, haddock, chub mackerel, mackerel, eel, sea perch, jack mackerel, sardine, perch, plaice, sole, flounder, cuttlefish, halibut, hake, megrim, swordfish, anchovy, pike and carp. In some embodiments the species of fish is selected from one or more of the group consisting of cod, herring, plaice and mackerel. In some embodiments the species of fish is cod. In some embodiments the fish protein comprises one or more of the proteins selected from the list consisting of Gad c 1, Clu h 1 and Ras k 1.

### Crustacean and Shellfish Protein

The most recent prevalence data from Asia highlight seafood as a significant sensitizer in up to 40% of children and 33% of adults (Lopata, A. L. and Lehrer, S. B., 2009. Current opinion in allergy and clinical immunology, 9(3), pp. 270-277.). Early introduction of crustacean or shellfish may decrease the risk of developing crustacean or shellfish allergy (Fleischer, D. M., et al., 2013. The Journal of Allergy and Clinical Immunology: In Practice, 1(1), pp. 29-36).

Crustacean and shellfish allergens are known in the art, for instance they are described in Lopata, A. L., et al., 2010. Clinical & Experimental Allergy, 40(6), pp. 850-858 and provided by the WHO/I UIS Allergen Nomenclature Database.

In some embodiments the species of crustacean is selected from one or more of the group consisting of crab, lobster, prawn and shrimp. In some embodiments the species of shell fish is selected from one or more of the group consisting of abalone, snail, whelk, clam, oyster, scallop, mussel, cockles, squid, octopus. In some embodiments the crustacean protein comprises one or more of the proteins selected from the list consisting of tropomyosin (Cha f 1, Cra c 1, Exo e 1, Hom a 1, Lit v 1, Pans 1, Mac r 1, Mel l 1, Mete 1, Pan b 1, Pen i 1, Pen m 1, Por p 1Pro c 1), Myosin light chain 2 (Hom a 3), Troponin C (Hom a 6). In some embodiments the shellfish protein comprises one or more of the proteins selected from the list consisting of Hal m 1 and Tropomyosin (Hel as 1, Crag 1, Sac g 1, Tod p 1).

### Sesame Protein

Allergy to sesame, often considered an emerging allergen, has been estimated to affect 0.10 to 0.79% of children from studies outside of the United States (Sicherer, S. H., et al., 2010. Journal of Allergy and Clinical Immunology, 125(6), pp. 1322-1326). Early introduction of sesame may decrease the risk of developing sesame allergy. For instance, it was selected as an allergen during the EAT study (Perkin, M. R., et al., 2016. Journal of Allergy and Clinical Immunology, 137(5), pp. 1477-1486).

Sesame allergens are known in the art, for instance they are described in Beyer, K., et al., 2002. Journal of Allergy and Clinical Immunology, 110(1), pp. 154-159. and provided by the WHO/IUIS Allergen Nomenclature Database.

In some embodiments the species of sesame is Sesamum indicum. In some embodiments the sesame protein comprises one or more of the proteins selected from the list consisting of 2s albumins (Ses i 1, 2); 7S vicilin-like globulin (Ses i 3), oleosin (Ses i 4, 5) and 11S globulins (Ses i 6, 7).

### Heat Treatments

The composition of the invention has preferably undergone gentle heat treatment, e.g. gentle pasteurisation and/or sterilisation. Reduced holding temperatures and/or holding times during heat treatment can reduce the extent of denaturation of the allergens.

“Pasteurisation” refers to partial sterilisation of a substance and especially a liquid (such as milk). Standard pasteurisation conditions will be well known to those of skill in the art, for example standard High Temperature Short Time (HTST) pasteurisation is typically used for milk pasteurisation, at a temperature of about 72° C. for 15 seconds. The temperature of pasteurisation is also known as the holding temperature and this temperature will be constant for the holding time.

In preferred embodiments the drinkable infant composition has undergone gentle pasteurisation thereby reducing thermal damage e.g. allergen denaturisation during pasteurisation. Gentle pasteurisation may be achieved using reduced holding temperatures and/or holding times. Thus, in some embodiments the pasteurisation is at a holding temperature of between 72° C. and 61.9° C., between 70° C. and 61.9° C., between 68° C. and 61.9° C., between 66° C. and 61.9° C., between 65° C. and 61.9° C. between 64° C. and 61.9° C., for example about 63° C.

Suitable holding times may be at least 15 minutes, 20 minutes, 25 minutes, 30 minutes or 35 minutes. For example, the pasteurisation may take place between 72° C. and 61.9° C. for between 15 and 45 minutes, or between 65° C. and 61.9° C. for between 20 and 40 minutes, preferably about 63° C. for about 35 minutes.

In some embodiments the gentle heat treatment may be carried out a temperature of between 72° C. and 90° C., for example between 72° C. and 80° C. for 10 to 30 seconds, or between 80° C. and 89° C. for 2 to 20 seconds, for example between 80° C. and 84° C. for 4 20 seconds, or between 85° C. and 89° C. for 1 to 10 seconds.

In some embodiments the drinkable infant composition, or the milk protein containing component, optionally undergoes microfiltration, prior to the gentle heat treatment, e.g. gentle pasteurisation. Suitable microfiltration techniques are well known in the art. The use of a microfiltration step can advantageously reduce bacterial load prior to the pasteurization step and can permit use of lower temperatures and/or holding times. In some embodiments of the present invention the drinkable infant composition has undergone sterilisation by ultra-high temperature (UHT) heat treatment. In preferred embodiments the sterilisation conditions are chosen such that they minimise thermal damage e.g. allergen denaturisation. This may be achieved by using reduced holding temperatures and/or holding times.

In some embodiments of the present invention the UHT heat treatment is an indirect heat treatment. An indirect heat treatment uses a heat-exchanger to elevate the liquid being sterilised to the holding temperature. Indirect UHT heat treatment can be used to minimise thermal damage by reducing the holding temperature, for instance as described in U.S. Pat. No. 4,534,986A. In some embodiments of indirect UHT heat treatment the holding temperature is between 125° C. and 135° C., or between 130° C. and 134° C., or between 131° C. and 133° C. In some embodiments of indirect UHT heat treatment the holding time is at least 30 seconds, or at least 60 seconds, for example between 30 and 80 seconds, or between 60 and 75 seconds. For example, an indirect UHT heat treatment is carried out between 131° C. and 133° C. for between about 60 and 75 seconds.

In some embodiments of the present invention the UHT heat treatment is a direct heat treatment. In direct heat treatment super-heated steam is mixed (e.g. injected) directly into the liquid. Direct heating may involve shorter times, which may also minimise thermal damage. In some embodiments the direct UHT heat treatment is at a temperature of between 136° C. and 140° C., for about 15 to 25 seconds, or at a temperature of between 140° C. and 144° C., for about 5 to 10 seconds, or at a temperature of between 150° C. and 154° C., for about 2 to 4 seconds.

In some embodiments the sterilisation is an ultra short sterilization (USS) heat treatment at a temperature of between 155° C. and 170° C., for less than 1 second.

In some embodiments of the present invention, following thermal sterilisation the drinkable infant composition is aseptically packaged.

In some embodiments of the present invention at least 20%, preferably at least 30%, of the two or more food allergens in the pasteurised nutritional composition according to the present invention are non-denatured. A high degree of non-denatured food allergens may be as the result of the heat treatments with reduced holding temperatures and/or holding times described herein.

In some embodiments of the present invention at least 20%, preferably at least 30%, of the milk protein is non-denatured. In some embodiments of the present invention at least 40% of the milk protein is non-denatured. In some embodiments of the present invention at least 50% of the milk protein is non-denatured.

In some embodiments at least 30% of each of the two or more food allergens is non-denatured. For example, between 30% and 100%, or between 30% and 95%, or between 30% and 90%, or between 40% and 80%, or between 50% to 60% of the food allergens are non-denatured.

According to the present invention, “denatured” proteins are proteins in which tertiary structures of the protein are disrupted or destroyed. Thus, typically in denatured proteins one or more of the interactions consisting of hydrogen bonding, salt bridges, disulphide bonds and non-polar hydrophobic interactions, are disrupted. “Denatured” proteins typically have the primary structure (i.e. peptide bonds) intact.

The percentage of food allergens that are non-denatured may be determined by any method known to those of skill in the art, for example, High Pressure Liquid Chromatography (HPLC), Fast Protein Liquid Chromatography (FPLC), Bicinochoninic Acid Assay (BCA), Kjeldahl Nitrogen (KN), Circular Dichroism (CD), Native-Polyacylamide Gel Electrophoresis (PAGE), Capillary Electrophoresis (CE), Fourier-Transform Infrared Spectoscopy (FTIR) or Fluorescence Spectroscopy.

If the nutritional composition contains more than one food allergen, each food allergen may be separated and analysed individually. The food allergens may be separated and analysed together. In some embodiments the average (mean) of all food allergens is non-denatured to the extent as specified herein.

Any method known to those of skill in the art may be used to separate the food allergens from the nutritional composition. For example, HPLC, FPLC, size exclusion chromatography, hydrophobic interaction chromatography, ion exchange chromatography, free-flow electrophoresis, affinity chromatography. These are described, for instance by Scopes, R. K., 2013. Protein purification: principles and practice. Springer Science & Business Media. In HPLC the separation of compounds is possible based on their interaction with the stationary phase. Common HPLC techniques include reverse-phase partition HPLC (RP-HPLC) and size exclusion HPLC.

In some embodiments of the present invention the Rowland method is used to determine the percentage of food allergens that are non-denatured, preferably wherein the food allergen is milk allergen.

In the Rowland method the non-denatured whey protein nitrogen (serum protein nitrogen, SPN) is defined as the nitrogen that is not precipitated by acetic acid and sodium acetate (Non Casein Nitrogen, NCN) minus the Non Protein Nitrogen (NPN), where SPN=NCN−NPN (Rowland, S. J., 1938. 175. Journal of Dairy Research, 9(1), pp. 30-46 and Rowland, S. J., 1938. 176). The NCN and total nitrogen (TN) is determined by the Kjeldahl method. NCN is determined from the filtrate after precipitation of the SPN.

For milk allergen the amount of non-denatured milk allergen protein may thus be expressed as the Serum protein nitrogen (non-denatured whey protein nitrogen) “SPN” as a percentage of total protein. SPN (as a % of total protein)=((NCN−NPN)/(TN−NPN))×100.

In some embodiments of the present invention Kjeldahl nitrogen is used to determine the percentage of food allergens that are non-denatured. Kjeldahl nitrogen is a well known method to determine the extent of protein denaturation (Parris, N. and Baginski, M. A., 1991. Journal of Dairy Science, 74(1), pp. 58-64).

In some embodiments of the present invention at least 20%, preferably at least 30%, of the milk protein is non-denatured.

### Prevention of Food Allergies

The present invention provides a drinkable infant composition as described herein for use in reducing or preventing food allergies in infants, particularly allergies to milk protein, egg protein, wheat protein, soya protein, peanut protein, tree nut protein, fish protein, crustacean protein, shellfish protein, and sesame protein.

The present invention also provides a method of reducing or preventing food allergies in infants by administering an effective amount of a drinkable infant composition as described herein.

In some embodiments an allergic response is a specific IgE-associated immune response and/or a T cell-dependent hypersensitive reaction. Thus, in some embodiments reducing or preventing allergies comprises reducing or preventing specific IgE-associated immune responses and/or a T cell-dependent hypersensitive reaction.

The drinkable infant composition may comprise milk protein for preventing or reducing allergy to milk.

The drinkable infant composition may comprise egg protein for preventing or reducing allergy to eggs.

The drinkable infant composition may comprise wheat protein for preventing or reducing allergy to wheat.

The drinkable infant composition may comprise soya protein for preventing or reducing allergy to soya.

The drinkable infant composition may comprise peanut protein for preventing or reducing allergy to peanut.

The drinkable infant composition may comprise tree nut protein for preventing or reducing allergy to tree nut.

The drinkable infant composition may comprise fish protein for preventing or reducing allergy to fish.

The drinkable infant composition may comprise crustacean protein for preventing or reducing allergy to crustacean.

The drinkable infant composition may comprise shellfish protein for preventing or reducing allergy to shellfish.

The drinkable infant composition may comprise sesame protein for preventing or reducing allergy to sesame.

In an embodiment, the prevention or reduction of food allergies in infants may also include induction of cross-tolerance, reducing or preventing development of allergy to a food allergen other than the allergens included in the drinkable infant composition.

Prior to administering the composition of the invention, a step of assessing the infant's risk of developing said food allergies may be carried out. This may comprise administering a small amount of allergen to the skin of relatives and/or a questionnaire for relatives. For example, an infant who has a parent or older sibling who has a food allergy may be at a greater risk of developing food allergy, therefore a drinkable infant composition comprising said food allergen may be administered to prevent or treat said food allergy.

### Processes

The drinkable infant composition according to the present invention may be prepared in any suitable manner. For example, a composition may be prepared by blending together the food allergens in appropriate portions, optionally blended with one or more carriers, such as an amino acid based infant formula, and then mixing the dry blended mixture with water to form a liquid mixture. The liquid mixture is then agitated for homogeneity. The temperature is then raised progressively and pasteurization is performed. The liquid mixture is then optionally spray-dried if the final product is to be a powder. The composition may be homogenised before pasteurisation or after pasteurisation.

In one aspect the present invention provides a process for producing the drinkable infant composition comprising the steps:
i) blending two or more food allergens wherein one of said allergens is milk protein to form a mixture;
ii) homogenising said mixture;
iii) pasteurising said mixture;
iv) optionally spray-drying said mixture;
   preferably wherein pasteurisation is a gentle pasteurisation as discussed herein.

In preferred embodiments pasteurisation is performed at a temperature of between 61.9° C. and 65° C., preferably between 62° C. and 64° C., preferably wherein the pasteurisation is performed for at least 30 minutes or at least 35 minutes. In one embodiment, the pasteurisation is performed at about 63° C. for between 30 minutes and 35 minutes.

In some embodiments the liquid mixture is homogenised then pasteurised. In other embodiments the liquid mixture is pasteurised then homogenised.

In one aspect the present invention provides a process for producing the drinkable infant composition comprising the steps:
i) blending two or more food allergens wherein one of said allergens is milk protein to form a mixture;
ii) homogenising said mixture;
iii) sterilising said mixture;
iv) optionally spray-drying said mixture;
   preferably wherein the sterilising is as discussed herein.

Preferably the homogenised liquid mixture is dried e.g. spray dried when the drinkable infant composition is in a powder form.

All publications mentioned in the above specification are herein incorporated by reference.

Various modifications and variations of the disclosed methods, processes, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

Preferred features and embodiments of the invention will now be described by way of non-limiting examples.

### EXAMPLES

### Example 1—an Infant Supplement

A drinkable infant supplement for clinical study purposes is provided. In this example the supplement contains milk protein and egg white protein combined with an amino-acid based infant formula (Alfamino infant formula (Nestlé)).

The supplement composition by weight (based on 3.1% moisture in the final product):
Skimmed milk powder 43.2%
Alfamino (amino-acid based hypoallergenic infant formula) 37.3%
Egg white powder 16.4%

Processing of the composition is by standard processing steps including homogenisation at 120/30 bar, gentle pasteurisation. Optionally the process may comprise spray drying to provide a powdered composition ready for reconstitution.

Pasteurisation is carried out at a holding temperature of 63° C. for a holding time of 2100 seconds to ensure the egg protein is not significantly damaged. A critical limit (i.e. minimum holding temperature) is set at 61.9° C. to ensure safety.

### Example 2—Gentle Pasteurisation

Gentle pasteurisation as defined above was carried out on a supplement containing milk protein. The level of denaturation was measured using the Rowland method.

The level of denaturation is expressed as Serum protein nitrogen (non-denatured whey protein nitrogen) “SPN” as a percentage of total protein=((NCN−NPN)/(TN−NPN))×100.

NCN=non casein nitrogen; NPN=non-protein nitrogen, TN=total nitrogen.

Before the heat treatment the level of non-denatured milk protein was 45%.

After gentle pasteurisation the level of non-denatured proteins was decreased to 36%. Therefore significant amounts of non-denatured milk protein remained.

### Example 3—Effect of Heat Treatment on Protein Degradation

Raw skimmed milk (24% total solids) was subjected to (i) warm microfiltration (14 μm filter at 52° C.) and the permeate was subjected to heat-treatment by direct steam injection (DSI) at 83° C. for 6 seconds, followed by spray-drying to form a powder “Prototype A”, (ii) cold microfiltration (14 μm filter at 15° C.) and the permeate was subjected to heat-treatment by direct steam injection (DSI) at 83° C. for 6 seconds, followed by spray-drying to form a powder “Prototype B”, and (iii) gentle pasteurisation at 63° C. for 35 minutes (as defined in Example 1), followed by spray drying “Prototype C”.

For the preparation of heat-treated samples 1-16 (Table 1) raw (unprocessed) milk (9-10% TS) was subjected to the selected heat treatments, as described in table 1, with or without homogenization before heat treatment. The homogenized milk variants (homogenisation at 150/30 bar) were spray-dried to get the final milk powders.

The level of protein denaturation of the Prototypes A, B and C, and heat-treated milk samples (heat-treatments as shown in Table 1) were measured using the Rowland method. The level of denaturation is expressed as Serum protein nitrogen (non-denatured whey protein nitrogen) “SPN” as a percentage of total protein, as in Example 2. Denaturation rate is expressed as the percentage of denatured whey proteins in the total proteins.

Native whey proteins (SPN) %=((NCN−NPN)/TN)*100

Whey protein denaturation (%)=100−[100*SPN]/[(TN−NPN)*0.2],

where [(TN−NPN)*0.2] is the total amount of whey protein nitrogen taking the whey:casein weight ratio in milk as 1:4.

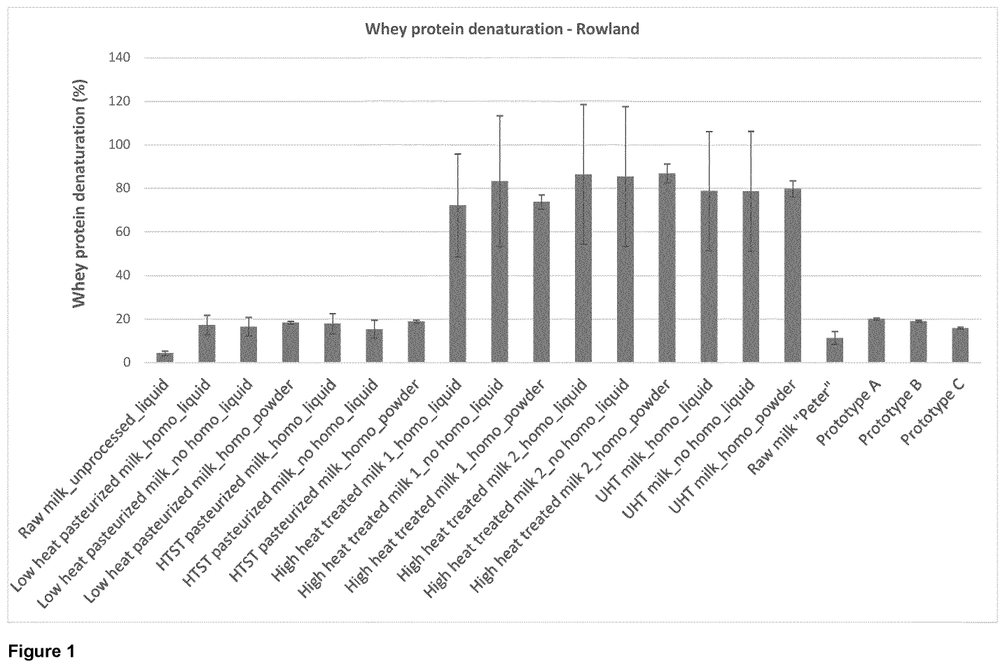

Results are shown in FIG. 1.

TABLE 1

| | | | Protein |
|---|---|---|---|
| | | | content |
| No. | Sample name | Process applied | (%) |
| 1. | Raw milk_unprocessed_liquid | Raw milk | 3.4 |
| 2. | Low heat pasteurized milk_homo_liquid | Homogenization + 63° C./30 min | 3.4 |
| 3. | Low heat pasteurized milk_no homo_liquid | 63° C./30 min | 3.4 |
| 4. | Low heat pasteurized milk_homo_powder | Homogenization + 63° C./30 min + spray drying | 34 |
| 5. | HTST pasteurized milk_homo_liquid | Homogenization + 72° C./15 s | 3.4 |
| 6. | HTST pasteurized milk_no homo_liquid | 72° C./15 s | 3.4 |
| 7. | HTST pasteurized milk_homo_powder | Homogenization + 72° C./15 s + spray drying | 34 |
| 8. | High heat treated milk 1_homo_liquid | Homogenization + 80° C./20 min | 3.4 |
| 9. | High heat treated milk 1_no homo_liquid | 80° C./20 min | 3.4 |
| 10. | High heat treated milk 1_homo_powder | Homogenization + 80° C./20 min + spray drying | 34 |
| 11. | High heat treated milk 2_homo_liquid | Homogenization + 90° C./20 min | 3.4 |
| 12. | High heat treated milk 2_no homo_liquid | 90° C./20 min | 3.4 |
| 13. | High heat treated milk 2_homo_powder | Homogenization + 90° C./20 min + spray drying | 34 |
| 14. | UHT milk_homo_liquid | Homogenization + 145° C./5 s | 3.4 |
| 15. | UHT milk_no homo_liquid | 145° C./5 s | 3.4 |
| 16. | UHT milk_homo_powder | Homogenization + 145° C./5 s + spray drying | 34 |

From FIG. 1 it can be seen that High-heat, high-time treatments (80, 90° C./20 min) and UHT treatments led to the highest levels of whey protein denaturation. Low heat-pasteurized milk samples, HTST (high temperature short time) pasteurized milk samples and prototypes A-C had similar denaturation profiles.

### Example 4—Immunological Activity

Immunological activity of the milk samples of Example 4 was assessed using a Humanised Rat Basophil Leukemia (RBL) degranulation assay (Bioceros Holding BV).

High affinity human FcεRI α chain expressing RBL cells were sensitized with an oligoclonal pool of chimeric (i.e., mouse variable IgG heavy and light domains combined with human constant IgE heavy and light chains, described in Knipping & Simons, PLoS ONE 2014; 9(8): e106025) human IgE Abs directed against BLG followed by exposure to either of the bovine milk samples (containing BLG) at different concentrations (0, 0.0032, 0.016, 0.08, 0.4, 2, 10, 100, 1000, 10000 ug/mL protein). RBL degranulation was determined by measuring extracellular β-hexosaminidase activity.

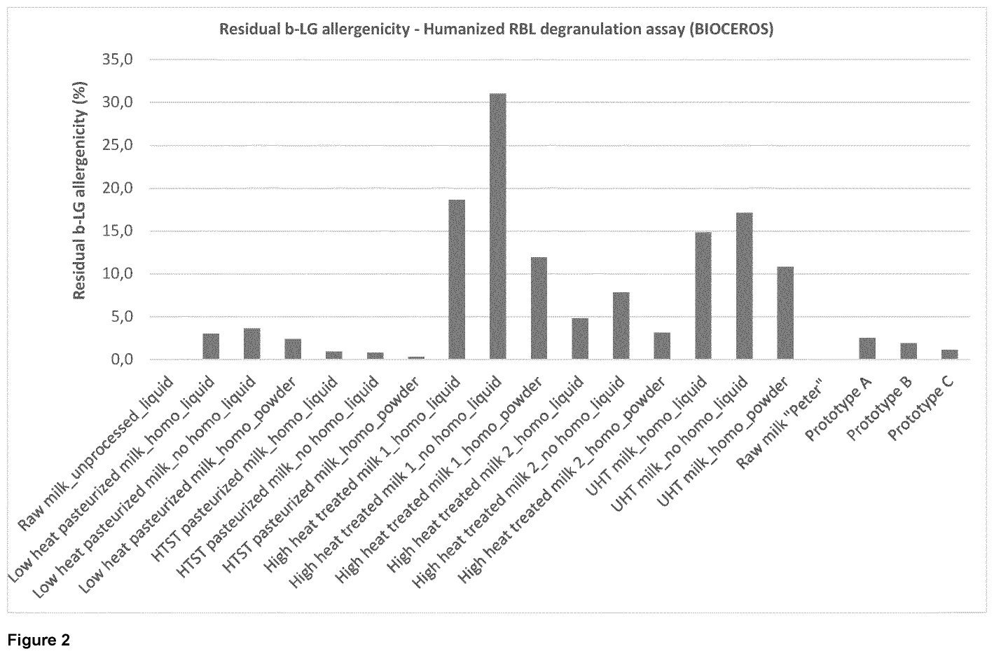

Results are shown in FIG. 2 and Table 2.

TABLE 2

| | | | % Residual BLG/ |
|---|---|---|---|
| Sample | Heat treatment | | Initial BLG |
| Raw milk_unprocessed_liquid | Raw milk | | 0.0 |
| Low heat pasteurized | 63° | C./30 min | 3.0 |
| milk_homo_liquid | | | |
| Low heat pasteurized milk_no | 63° | C./30 min | 3.6 |
| homo_liquid | | | |
| Low heat pasteurized | 63° | C./30 min | 2.4 |
| milk_homo_powder | | | |
| HTST pasteurized | 72° | C./15 s | 0.9 |
| milk_homo_liquid | | | |
| HTST pasteurized milk_no | 72° | C./15 s | 0.8 |
| homo_liquid | | | |
| HTST pasteurized | 72° | C./15 s | 0.3 |
| milk_homo_powder | | | |
| High heat treated milk | 80° | C./20 min | 18.6 |
| 1_homo_liquid | | | |
| High heat treated milk 1_no | 80° | C./20 min | 31.0 |
| homo_liquid | | | |
| High heat treated milk | 80° | C./20 min | 11.9 |
| 1_homo_powder | | | |
| High heat treated milk | 90° | C./20 min | 4.8 |
| 2_homo_liquid | | | |
| High heat treated milk 2_no | 90° | C./20 min | 7.8 |
| homo_liquid | | | |
| High heat treated milk | 90° | C./20 min | 3.1 |
| 2_homo_powder | | | |
| UHT milk_homo_liquid | 145° | C./5 s | 14.8 |
| UHT milk_no homo_liquid | 145° | C./5 s | 17.1 |
| UHT milk_homo_powder | 145° | C./5 s | 10.8 |

| | | |
|---|---|---|
| Prototype A | Warm microfiltration (1.4 um, | 2.5 |
| | 52° C.) + Direct Steam Injection | |
| | (83° C., 6 s) + spray drying | |
| Prototype B | Cold microfiltration (1.4 um, | 1.9 |
| | 15° C.) + Direct Steam Injection | |
| | (83° C., 6 s) + spray drying | |
| Prototype C | LTLT pasteurization (63° C./30 | 1.1 |
| | min) + spray drying | |

### Example 5—Maintenance of Immunogenic Potential

### A. SDS-PAGE

The infant supplement of Example 1 “PREMEA” was analysed by gel electrophoresis (SDS-PAGE). Storage was carried out at 4° C., 25° C. and 37° C. for 6 months. Gel electrophoresis was carried out using a Novex NuPAGE® system (Thermo Fisher Scientific), following the method protocol as provided by the manufacturer. The employed separation gel was a precast NuPAGE® 4-12% Bis-Tris gel in combination with the MES SDS running buffer.

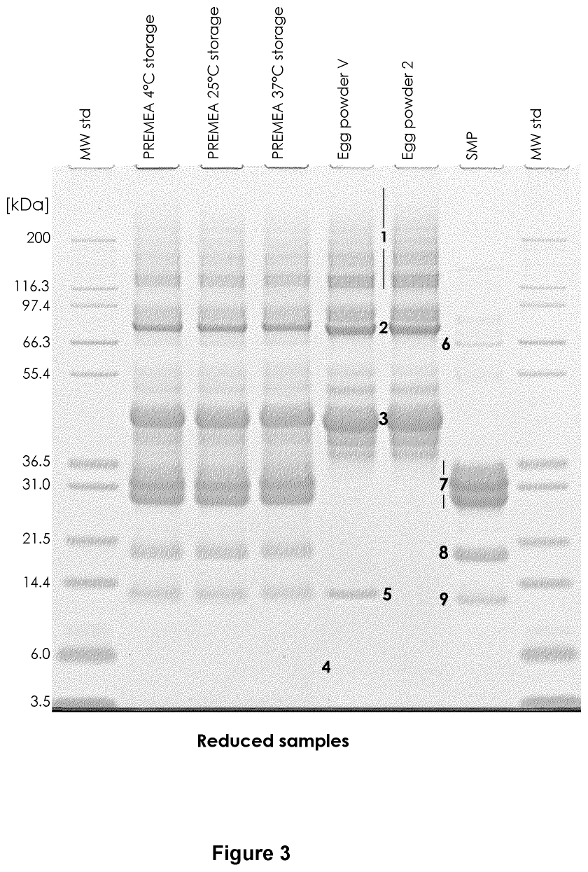

Results are shown in FIG. 3. The band assignment (bands 1-9) in FIG. 3 identified the following protein bands:

### Egg White Powder

1. Ovomucins (<100 kDa)
2. Conalbumin (77.7 kDa)
3. Ovalbumin (44.5 kDa)
4. Ovomucoid (30-40 kDa)
5. Lysozyme (14.3 kDa)

### Skim Milk Powder

6. Serum albumin (66 kDa)
7. Caseins (28-35 kDa)
8. β-lactoglobulin (18.8 kDa)
9. α-lactalbumin (14.4 kDa)

Conclusions: Protein bands from egg and milk are found in the prototype. Results demonstrate that the main milk and egg proteins (including the most allergenic ones such as ovomucoid or beta-lactoglobulin) are not degraded by manufacturing processing of the final product. The band intensities of milk and egg allergens for the prototype indicate their significant quantities in the prototype.

### B. Quantification of Ovomucoid

Egg white contains 23 different glycoproteins. Among them, ovomucoid (Gal d 1) comprises approximately 11% of the total egg white protein, and has been shown to be the dominant allergen of egg (1, 2). To compare immunogenicity of the proteins present in the infant supplement of after processing to raw material used, quantified ovomucoid as a representative allergen of egg.

### Method:

Ovomucoid was detected and quantified using a commercialized ELISA Kit (BioKits Egg Assay Kit; Neogen corporation, USA), ref 902072T), according to the Manufacturer's instructions. The polyclonal antibody (significantly more robust when assaying proteins that show slight variations in individual epitopes such as denaturation, polymorphism or conformational change) used in this kit specifically detects ovomucoid (Gal d1).

The egg white raw material used for the manufacturing of the prototype, as well as the prototype, stored for 6 months at different temperatures (4° C., 25° C. and 37° C.) were analyzed. The quantity of ovomucoid measured in the prototype was then calculated back according to the % of egg material present in the prototype to enable direct comparison between the raw material and the final product.

### Results:

Ovomucoid content in egg white is 205.9±24.7 mg/g protein. Similar ovomucoid content was quantified in the prototype stored at 4° C., 25° C. and 37° C. (158.3±43.5; 175.8±42.7 and 222.1±20.9 mg/ml, respectively).

### Conclusion:

Using a quantitative method, no significant difference in immunogenicity (epitopes recognized by polyclonal antibody against ovomucoid) between the egg white raw material and the final processed product, stored for 6 months at 4° C., 25° C. and 37° C., was observed. These results demonstrate that ovomucoid in the sample was not degraded on storage.

### Example 6—Quantification of Beta-Lactoglobulin and Ovalbumin

Three samples of a mixture of raw (unprocessed milk) and raw egg white were prepared by mixing 3 volumes of milk with 1 volume of egg white. Protein content calculated as egg white proteins 100 g/L, milk proteins 33 g/L. The samples were pre-treated at 55° C. for 5 minutes before being subjected to gentle pasteurization under different conditions (i) 63° C. for 30 minutes; (ii) 70° C. for 3 minutes; (iii) 70° C. for 20 minutes. The samples had pH 6.7.

One set of samples of the infant supplement of Example 1 “PREMEA” and the milk/egg white samples (i)-(iii), at pH 6.7, were subjected to ultracentrifugation (10000 g/1 h) for removing aggregated proteins. Quantification of the native proteins and soluble denatured proteins was carried out by protein separation by gel electrophoresis (SDS-PAGE) according to Example 5, followed by protein quantification by densitometric scanning of the intensity of the stained electrophoretic bands.

A second set of samples of the infant supplement of Example 1 “PREMEA” and the milk/egg white samples (i)-(iii), was acidified to pH 4.6, followed by centrifugation at 14000 g/10 min) for removing precipitate (denatured and aggregated proteins). Quantification of the native proteins was carried out as above.

Quantification of the denaturation level of beta-lactoglobulin and ovalbumin is expressed as Denaturation rate (%)=[1−(I_{pH4.6}/I_{pH6.7})], where I_{pH4.6} is the native protein concentration and I_{pH6.7} is the total protein concentration (native+soluble denatured protein).

Results are shown in Tables 3.4 and 5.

### Milk/Egg White Protein Denaturation

TABLE 3
Beta-lactoglobulin

| | Ratio intensity |
|---|---|
| Sample | pH 4.6/pH 6.7 |
| PREMEA | 0.35 |
| 63° C./30 min | 0.69 |
| 70° C./3 min | 0.60 |
| 70° C./20 min | 0.46 |

TABLE 4
Ovalbumin

| | Ratio intensity |
|---|---|
| Sample | pH 4.6/pH 6.7 |
| PREMEA | 0.51 |
| 63° C./30 min | 0.77 |
| 70° C./3 min | 0.67 |
| 70° C./20 min | 0.68 |

### Milk/Egg White Protein Denaturation

TABLE 5

| | Level of denaturation | Level of denaturation |
|---|---|---|
| Sample | beta-lactoglobulin | Ovalbumin |
| PREMEA | 0.64 | 0.49 |
| 63° C./30 min | 0.31 | 0.23 |
| 70° C./3 min | 0.4 | 0.33 |
| 70° C./20 min | 0.54 | 0.32 |

1. A drinkable infant composition comprising two or more food allergens from different foods wherein one of said food allergens is milk protein.

2. A drinkable infant composition according to claim 1 wherein the composition a powder for reconstitution in a liquid, before administration.

3. A drinkable infant composition according to claim 1 wherein the composition is in a ready to drink form.

4. A drinkable infant composition according to claim 1, wherein the composition has undergone pasteurisation at a temperature of between 61.9° C. and 65° C.

5. A drinkable infant composition according to claim 1 wherein the composition has undergone thermal sterilisation.

6. A drinkable infant composition according to claim 5 wherein the thermal sterilisation is an indirect ultra-high temperature heat treatment.

7. A drinkable infant composition according to claim 1, wherein the food allergens are selected from the group consisting of: egg protein, wheat protein, soya protein, peanut protein, tree nut protein, fish protein, crustacean protein, shellfish protein, and sesame protein.

8. A drinkable infant composition according to claim 7 wherein the composition comprises three or more of said food allergens.

9. A drinkable infant composition according to claim 7, wherein the composition comprises milk protein and egg protein.

10. A drinkable infant composition according to claim 1, wherein the composition is for use in infants between 0-12 months.

11. A drinkable infant composition according to claim 1, wherein the total amount of said food allergens in the composition is between 0.5 and 5 grams per serving.

12. A drinkable infant composition according to claim 1, wherein at least 20% of said milk allergens are non-denatured.

13. A method for reducing or preventing allergies to said food allergens in infants comprising administering to the infant a drinkable infant composition comprising two or more food allergens from different foods wherein one of said food allergens is milk protein.

14. A process for producing a drinkable infant composition comprising the steps:
i) blending two or more food allergens wherein one of said allergens is milk protein to form a mixture;
ii) homogenising said mixture; and
iii) pasteurising said mixture.

15. (canceled)
